(19) United States
(12) Patent Application Publication
(10) Pub. No.: US 2005/0137452 A1
(43) Pub. Date: Jun. 23, 2005

(54) Device for covering a tissue region on a wall of a heart

(75) Inventor: Peter Osypka, Grenzach-Wyhlen (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Dr. Osypka GmbH, Rheinfelden (DE)

(21) Appl. No.: 11/018,450

(22) Filed: Dec. 20, 2004

(30) Foreign Application Priority Data
Dec. 19, 2003 (DE) 103 59 789.1-43

(51) Int. Cl. A61F 2/00

(52) U.S. Cl. 600/37

(57) ABSTRACT

A device (1) for covering, supporting, and/or sealing a region of tissue affected by a ventricular septal defect (2) in the heart wall (3) between the left and right ventricles is provided. It has at least one, and preferably two oblong holding and/or guiding parts (5), which are each used for at least one soft and smooth membrane body (6) or a comparable patch. The patch or membrane body (6) is longitudeinally stretched out against its elasticity in an original position and has only a small lateral extent relative to its length. By loosening one end of this longitudinally stretched-out arrangement, the membrane body (6) can be widened due to a restoring force, so that it attains a planar shape for covering the heart-wall defect (2).

### BACKGROUND

The invention relates to a device for covering, supporting, and/or sealing a region of tissue affected by a ventricular septal defect in the heart wall between the left and right ventricles.

A ventricular septal defect is one of the acute, life-threatening occurrences for humans. A hole in the heart wall between the left and right ventricles due to this defect cannot be treated by acute surgery, because the tissue is split and cannot be sewed together.

Therefore, attempts have been made to use devices developed for atrial septal defects for emergency aid, but for the most part these attempts have been without success. Only if the defect is so small that it allows an approximately two-week recovery phase, during which the patient remains alive, is there the possibility of surgery.

SUMMARY

Therefore, there is the objective of creating a device of the kind mentioned above, which allows the time period of a recovery phase to be bridged, without the risk during this time that the circulation of the patient will fail with the result of death due to blood crossing from the left to right ventricle.

To achieve this object, the device according to the invention includes an oblong holding and/or guiding part for at least one soft and/or smooth membrane body, which is longitudinally stretched out in an original position and has a small lateral extent relative to its length and is arranged along the guiding part and fixed to this guiding part at two spaced attachment points. A restoring force acts on the membrane body in its longitudinally stretched-out position, and by means of this force, the membrane body is shortened and widened after being detached from at least one attachment point and has a planar shape for covering the heart-wall defect.

Such a device can be introduced surgically adjacent to the heart wall approximately at the tip of the heart into the interior of the heart, with a very small incision being sufficient, because the guiding part and the membrane body have a small width. After introduction, an attachment point to the membrane body can be detached so that the membrane body widens due to the restoring force and therefore covers the heart-wall defect and can prevent blood crossover. Thus, it is possible with a small operation on the heart to obtain sufficient recovery time for the damaged tissue in the case of a ventricular septal defect. Here, the surgeon can insert the guiding part with a very small lateral extent with membrane body into the interior of the heart through a small incision, and then from outside the heart, the one attachment point of the membrane body can be loosened so that the membrane body expands and/or unfolds in the interior of the heart due to the restoring force acting on it.

Preferably, the membrane body has in its edge region an elastically flexible reinforcement, for example, in the form of at least one wire, which, in the non-tensioned position, has a loop-shaped or arc-shaped profile along the edge of the planar membrane and which, in the original position, can be deformed against an elasticity and restoring force such that two opposite sides or edges of the membrane body are brought close to reinforcing arc pieces of the reinforcement and are at least partially stretched or straightened, and/or that the membrane body is formed especially out of elastic material. Through a tensile force on the reinforced and/or elastic membrane body in an extension direction of the guiding part, the guiding part itself can have a narrow and long shape and it can be pretensioned, so that the entire device fits through a very small incision in the region of the tip of the heart and can be inserted into the heart. Then, if the tensile force is removed, the elastic membrane changes shape back into a planar body, which can be supported by elastic reinforcement, so that a correspondingly large damaged region of tissue in the heart wall can be covered, so that blood crossover is prevented. If sufficient recovery time has elapsed, the membrane body can be stretched-out longitudinally again and the device can be removed from the heart.

The flexible, especially wire-shaped reinforcement can be formed by two arc-shaped pieces, which preferably project out from a common holder located at a first attachment point of the guiding part and extend to a preferably common counter-guide, which can be adjusted on the guiding element in its longitudinal direction, which forms two attachment points, and whose spacing relative to the first attachment point can be made greater against the elasticity or restoring force of the reinforcements and/or the membrane, and reduced in the position of use by this restoring force. Thus, the device has on the guiding part two spaced attachment points, whose spacing can be changed. For a greater spacing, the membrane body is stretched and narrow or thin and thus can be easily inserted into the heart, after which, by moving the attachment points closer due to the restoring force, the lateral extent of the membrane can be made greater for covering the region of damaged tissue.

A tension element can be attached, especially detachably, to the adjustable counter-guide and this tension element can be used simultaneously for fixing the longitudinally stretched-out, narrow shape under a restoring force or the original position of the membrane body. Thus, this longitudinally stretched-out shape can be fixed detachably, because after the membrane body is introduced into the heart, only this tension element needs to be detached or loosened in order to let the restoring force become effective. For removing the device, the tension element can be tightened again, and this action brings the membrane body back into its narrow shape.

The guiding part can be a pin, rod or wire, and the counter-guide can be a ring or partial ring at least partially surrounding the guiding part. This represents an especially preferred and simple form of the invention, which simultaneously can have a very small width and thickness.

The tension element can be thread or wire, which has an especially U-shaped profile with two U legs and can be attached to these legs and which extends through an eyelet, a hook, or the like that is adjustable in common with the counter-guide near or on this adjustable counter-guide part. Thus, the tension element on the counter-guide requires less room and can be adjusted against the restoring force. If the tension element is to be removed, only one U leg can be pulled.

In another configuration of the invention for improving the support in the interior of the heart, the guide part is reinforced by at least one other parallel and/or at least section-wise curved brace, for example, a rod, rail, or the like, especially by such braces, rods, rails, or the like arranged on both sides of the guide part, with these braces extending in the position of use on the side of the membrane facing away from the heart wall and preferably engaging from behind or below and supporting this membrane. Here, the device does attain a somewhat greater width in the direction of the profile of the heart wall, as well as better support of the elastic membrane, so that it can be pressed onto the heart wall well accordingly.

The brace or braces can be arranged in a plane offset relative to the surface of the membrane body. In this way, the device becomes more resistant to bending.

An especially preferred and effective embodiment of the device is provided with two guiding parts, each with at least one membrane body, between which in the position of use the heart wall is arranged. The two guiding parts are connected to each other in the position of use outside of the heart such that they and their membrane body are pressed together and therefore are pressed onto the heart wall from both sides. In this way it can be achieved especially well that the defective part of the heart wall is supported and sealed, and despite the damage can withstand the blood pressure independent of its effective direction. In addition, each membrane can contact and even press better on the defective heart wall than would be possible with an individual guide with a membrane body.

The two guide parts can be connected like tweezers and are close to each other in the original position and can be spread apart against a restoring force for insertion in the position of use. Thus, based on the shaping of the device, they are pressed, so to speak automatically, from both sides onto the heart wall.

An especially preferred embodiment of a device with two membrane bodies provides that the two guiding parts, at a location outside of the region acted upon by the membrane bodies, in the position of use, are hinged by a connecting device in the region remaining or located outside of the heart, for example, with a ring or, preferably elastic, collar or the like, and can be spread apart on the side of this connecting device facing away from the membrane bodies and therefore the membrane bodies can be close to each other or can be pressed together. The two guiding parts can thus lie in the position of use outside of the heart relative to each other so that, for a spreading apart motion, the regions projecting over the contact position have the effect that the regions of the guiding parts located in the heart approach each other and therefore are pressed with their membrane body onto the heart wall.

In this way, at the regions of the guiding parts projecting over the connecting device or position, at least one spreading element for spreading open and fixing the spread position can be attached or formed, especially for increasing the spreading force and thus the pressure force acting on the membrane bodies can be shifted in the direction toward the connecting position. Thus, the device can be placed at first relatively loosely in the interior of the heart and then spread with the help of the spreading parts, so that the membrane bodies are pressed from both sides onto the defective heart wall.

It is still to be mentioned that the two guiding parts also cross outside the heart and could be connected by a hinge pin or the like, so that pressing together the overlapping ends also brings the membrane bodies closer to each other and presses them onto the heart wall. However, this means that the guiding parts cannot be operated on individually in the heart, as is possible in the arrangement with the help of a collar or a ring, which joins the two outer-lying regions and which is attached after the introduction of the guiding parts, or that the hinge pin must be attached at a later time after the operation on the guiding parts.

Above all, combining individual or several of the previously described features and means results in a device which allows a temporary closing of the opening between the two ventricles in the case of a ventricular septal defect, so that after a sufficient recovery time, this defect can then be operated on.

### BRIEF DESCRIPTION OF THE DRAWINGS

In the following, the preferred embodiments of the invention are described in more detail with reference to the drawings. In the drawings, shown in partially schematic illustration:

FIG. 1 is a section view through a human heart, for which the heart wall between the left and right ventricles has a ventricular septal defect, which is covered from both sides by a device according to the invention with two guiding parts and membrane bodies held by these parts;

FIG. 2 is an enlarged scale side view of a guiding part with a elongated membrane body mounted on the guiding part against a restoring force;

FIG. 3 is a top view of the device or the guiding part with the longitudinally stretched-out membrane body according to FIG. 2;

FIG. 4 is a view corresponding to FIG. 2 after the release of an attachment point, which therefore approaches the second attachment point;

FIG. 5 is a top view of the arrangement according to FIG. 4, with the membrane body being shortened and spread in two dimensions due to the restoring force, so that it has a shape provided for covering the ventricular septal defect;

FIG. 6 is a side view of a modified embodiment, for which the guiding part is a single rod or pin; and

FIG. 7 is a view similar to FIG. 5 of the embodiment according to FIG. 6.

### DETAILED DESCRIPTION OF THE PREFERRED EMBODIMENTS

A device according to the invention designated as a whole with 1 is used for covering, supporting, and/or sealing a region of tissue affected by a ventricular septal defect 2 in the heart wall 3 between the left and the right ventricles of the heart 4.

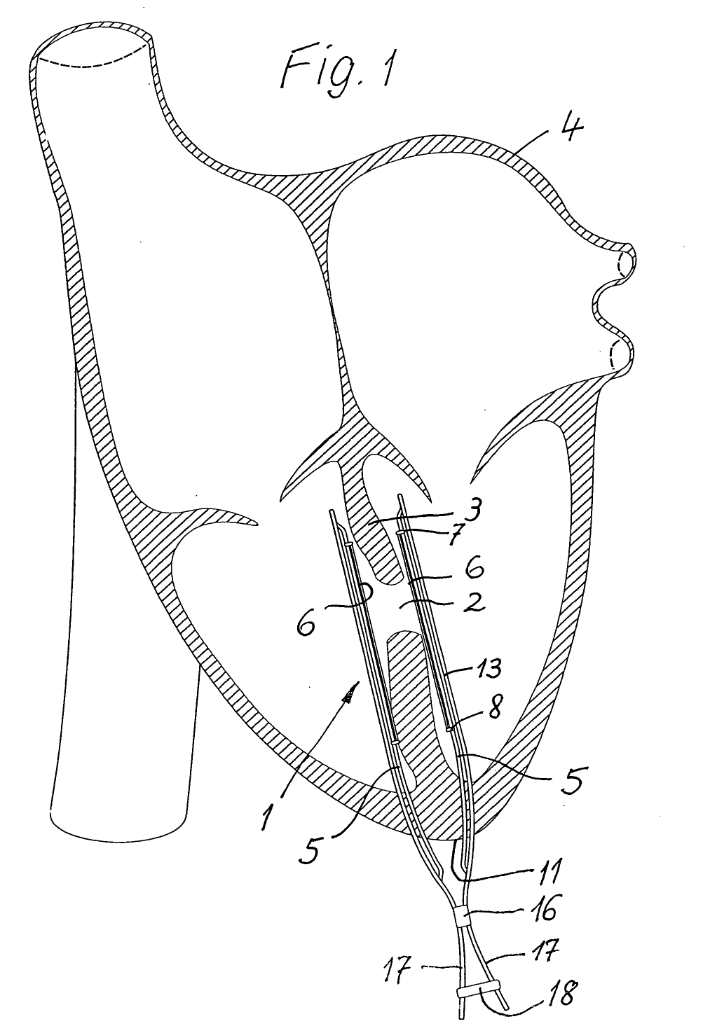

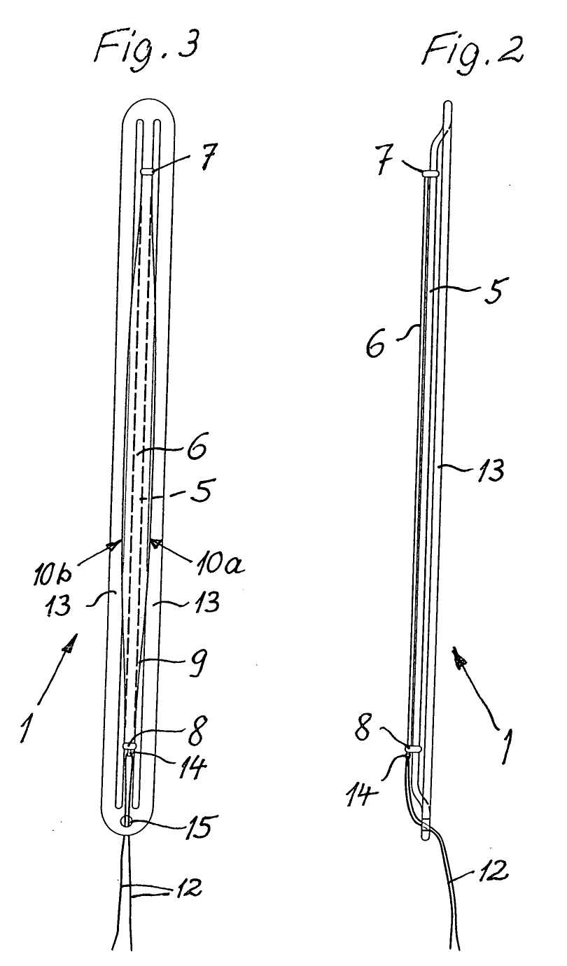

According to FIG. 1, two oblong holding and/or guiding parts 5 are each provided for at least one soft and smooth membrane body 6, which are shown even more clearly with reference to FIGS. 2 to 7 and are explained in the following.

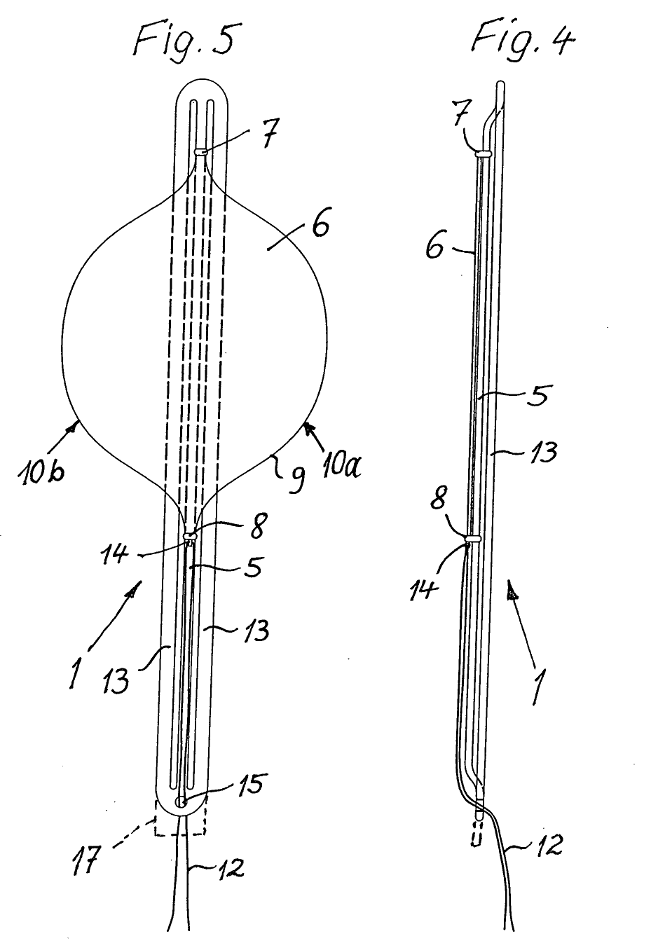

In FIGS. 2 and 3, one can see that the membrane body 6 is longitudinally stretched-out in the original position and has a small lateral extent relative to its length and is fixed along the guiding part 5 which supports it at two spaced attachment points 7 and 8, so that the membrane body 6 is arranged on the guiding part 5 and connected to it. In this longitudinally stretched-out position, a restoring force acts on the membrane body 6. Through this force, the membrane body is shortened and widened after being detached from at least one attachment point 8, according to FIGS. 4 to 7, with the widening being especially easy to see in FIGS. 5 and 7. The membrane body 6 thus attains a planar shape for covering the heart-wall defect 2.

Here, release from the attachment point 8 does not mean that the membrane body 6 is afterward no longer connected to the guiding part 5, instead by comparing FIGS. 2 and 3 with FIGS. 4 and 5 it becomes quite clear that the second attachment point 8 remains functional as such, but its spacing to the first attachment point 7 can be reduced, which can be brought about by the elasticity or restoring forces, when the attachment point 8 is released in a way still to be described for a corresponding displacement motion.

The membrane body 6 has an elastic, flexible reinforcement 9 in its edge region forming the edge of the membrane body, which in the preferred embodiment is in the form of at least one wire. This reinforcement 9 has a loop-shaped or arc-shaped profile in the non-tensioned position according to FIGS. 5 and 7 along the edge of the planar membrane 6 and can be deformed against an elasticity and restoring force such that, by becoming long and stretched out, two opposite sides or edges 10a and 10b of the membrane body 6 and thus the reinforcements 9 arranged there are close to each other and are stretched or straightened at least partially.

Instead or preferably in addition, the membrane body 6 itself can be formed of elastic material, which allows the planar membrane to become long and stretched out and which therefore assumes a smaller width.

The flexible, especially wire-shaped reinforcement 9 is formed in the embodiment by two arc-shaped pieces on the edges 10a on one side and 10b on the other side, which project from a common holder located on the first attachment point 7 of the guiding part 5 and extend to a counter-guide, which can be adjusted on the guiding part 5 in its longitudinal direction and which has or forms the second attachment point 8, and whose spacing relative to the first attachment point 7 can be made greater against the elasticity or restoring force of the reinforcements 9 and/or the membrane 6, and can be made smaller or reduced in the position of use by this restoring force.

Through these measures, at first the membrane body 6 can be brought into a longitudinally stretched-out position, in that the counter-guide holding it as attachment point 8 is brought into a greater spacing to the attachment point 7, by means of which the membrane is stretched out longitudinally against the restoring force and is offset in a narrow state. In this position shown in FIG. 3, the device can be preferably inserted into the heart through an incision near the tip 11 of the heart and applied to the heart wall 3, after which the holder of the attachment point 8 is detached or released and this point comes closer to the attachment point 7 due to the restoring force, which automatically has the effect of expanding the membrane body 6 into the position shown in FIGS. 5 and 7.

For this activation, a tension element 12 can be attached, especially detachably, to the adjustable counter-guide for the attachment point 8 and this tension element 12 can also be used for fixing the longitudinally stretched-out narrow shape under a restoring force or original position of the membrane body 6. Thus, after inserting the device 1 into the interior of the heart, only this tension element needs to be loosened or detached in order attain the planar shape of the membrane body 6 for covering the defect 2 in the interior of the heart.

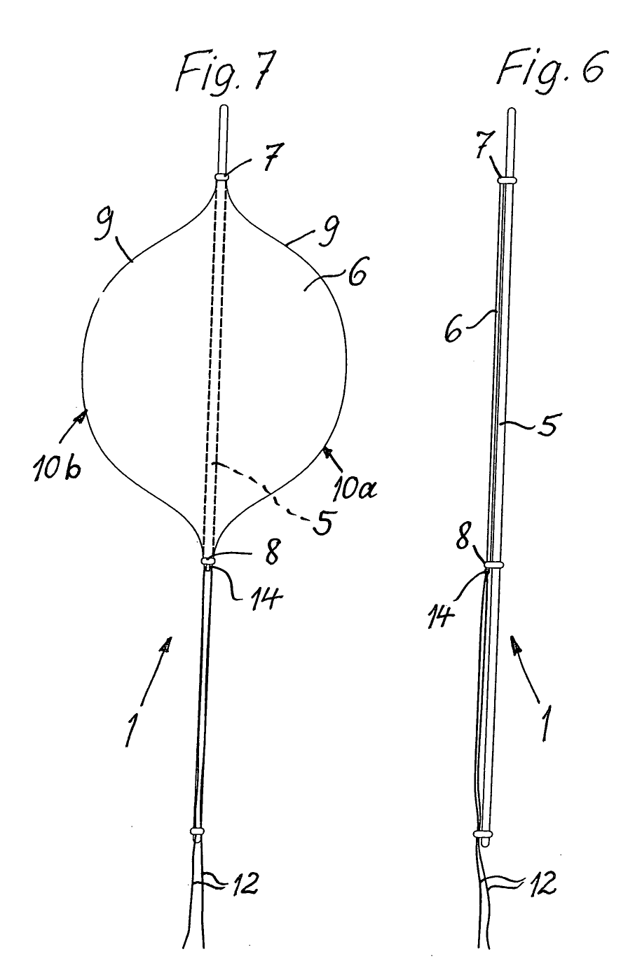

In FIGS. 6 and 7, another embodiment of the device 1 according to the invention is shown, for which the guiding part 5 is a pin or rod or wire, and the counter-guide is a ring or partial ring at least partially surrounding this guiding part. This produces a device 1 that is favorable primarily in the lateral dimensions, for which a correspondingly smaller incision is required near the tip 11 of the heart.

In FIGS. 2 to 5, a modified embodiment is shown, for which the guiding part 5 is reinforced by at least one, and as illustrated, two other parallel braces 13, which are arranged on both sides of the guiding part 5. These braces 13 extend on the side of the membrane 6 facing away from the heart wall 3 in the position of use and engage these from behind or below, so that they can form additional support primarily when the membrane 6 is pressed onto the heart wall 3.

In FIGS. 1, 2, and 4, one can still see that the braces 13 are arranged in a plane offset relative to the surface of the membrane body 6. In this way, in total an especially stiff structure is realized, formed by the braces 13 and the guiding part 5.

In FIGS. 2 to 7, one can still see that the tension element 12 is a thread or wire, which has somewhat of a U-shaped profile with two U legs and which can be fixed to these legs, for example, by links, and which extends through an eyelet 14 adjustable with the counter-guide near or on this adjustable counter-guide part forming the attachment point 8. By tensioning the tension element 13, the eyelet 14 and thus the adjustable counter-guide part forming the attachment point 8 can be brought from the position shown in FIGS. 4 and 5 or 6 and 7 into the tensioned position.

In addition, the tension element extends on the end facing away from the fixed attachment point 7 through an opening 15 on the side of the device 1 facing away from the heart wall 3 in the position of use, so that the tension element 13 is also guided and held.

As already mentioned, in the embodiment according to FIG. 1 the device 1 has two guiding parts 5, which each carry at least one membrane body 6 in the previously described way and between which the heart wall 3 with the defect 2 is arranged in the position of use.

According to FIG. 1, these two guiding parts 5 are connected to each other in the position of use outside of the heart 4 such that they and their membrane bodies 6 are pressed together and therefore pressed from both sides onto the heart wall 3, so that the defect 2 is supported and sealed correspondingly well. Thus, the tissue in the intermediate space can recover for a sufficiently long period of time, so that after this recovery time, the device 1 can be removed and the defect can be treated with surgery.

The two guiding parts 5 with the membranes 6 carried by them can be connected like tweezers and can be close to each other in the original position and can be spread apart for insertion in the position of use against a restoring force.

However, according to the embodiment from FIG. 1, it is more favorable if the two guiding parts 5 are hinged outside of the region acted upon by the membrane bodies 6, in the position of use at the region located outside of the heart 4, by means of a connecting device 16, in the embodiment with the help of a collar, and can be spread apart on the side of this connecting device 16 facing away from the membrane bodies 6, and therefore the membrane bodies 6 can be brought closer to each other or pressed together. With reference to FIG. 1, it can be clearly seen that when the two ends 17 of the guiding parts 5 are spread apart, the regions of these guiding parts 5 located in the heart are pressed together and therefore the membranes 6 are pressed onto the heart wall 3.

In this embodiment, a spreading element 18 is provided for spreading open and fixing the spread at the regions extending over the connecting device 16 of the guiding parts 5, thus at the ends 17. The spreading element 18 can be shifted for increasing the spreading force and thus the pressure force acting on the membrane bodies 6 in the direction towards the connecting position 16, so that due to the constant spacing of the holes passing from the ends 17 into the spreading element 18, these ends 17 can be pressed further apart, therefore increasing the pressure force of the two membranes 6 against the heart wall 3.

The device 1 is used for covering, supporting, and/or sealing a region of tissue affected by a ventricular septal defect 2 in the heart wall 3 between the left and the right ventricles. It has at least one, and preferably two, oblong holding and/or guiding parts 5, which are each used for at least one soft and smooth membrane body 6 or a comparable patch, with this patch or this membrane body 6 being longitudinally stretched out in the original position due to its elasticity and having only a small lateral extent relative to its length. Through loosening one end of this longitudinally stretched-out arrangement, the membrane body 6 can be widened due to a restoring force, so that it attains a planar shape for covering the heart-wall defect 2.

It is still to be mentioned that in FIGS. 2 to 7, the regions with the connecting device 16 or extensions 17 of the guiding parts (5) are not shown or are “broken off” for better clarity, but in reality they extend adjacent to the holes 15 on the projection of the guiding parts.

Furthermore, only one guiding part 5 with membrane body 6 can be used and can interact in the heart with one counter holder, which could be arranged at the position of the second guiding part 5 and could be equipped with an extension 17 in order to be able to press the membrane body 6, which is activated and held from the outside, in the interior of the heart 4.

1. Device (1) for covering, supporting, and/or sealing a region of tissue affected by a ventricular septal defect (2) in the heart wall (3) between the left and right ventricles, comprising at least one oblong holding and/or guiding part (5) for at least one soft and/or smooth membrane body (6), which is longitudinally stretched out in an original position and has a small lateral extent relative to a length thereof, and is arranged along the guiding part (5) and fixed to the guiding part at first and second spaced apart attachment points (7, 8), and after release of at least one of the attachment points (8), is moved to a use position by a restoring force (6) that acts on the membrane body (6) in which the membrane body is shortened and widened to have a planar shape for covering the heart-wall defect (2).

2. Device according to claim 1, wherein the membrane body (6) has an elastic, flexible reinforcement (9) in an edge region thereof comprising at least one wire, which has in a non-tensioned position a loop-shaped or arc-shaped profile along the edge region of the planar membrane (6), and can be deformed in the original position against an elasticity and restoring force thereof such that arc-shaped portions of the reinforcement (9) reinforcing two opposite sides or edges (10a, 10b) of the membrane body (6) are close to each other and are stretched or straightened at least partially.

3. Device according to claim 2, wherein the flexible reinforcement (9) is formed by two arc pieces, which project from a common holder located at the first attachment point (7) to the guiding part (5) and extend to a common counter-guide, which is adjustable on the guiding part (5) in a length direction and forms the second attachment point (8), and whose spacing relative to the first attachment point (7) can be made greater against the elasticity or restoring force of the reinforcement and/or the membrane (6), and in the use position is reduced by the restoring force.

4. Device according to claim 3, wherein a tension element (12) is connected to the adjustable counter-guide for fixing the longitudinally stretched-out narrow shape under a restoring force for the original position of the membrane body (6).

5. Device according to claim 1, wherein the guiding part (5) is a pin, rod or wire and the counter-guide is a ring or partial ring at least partially surrounding the guiding part.

6. Device according to claim 4, wherein the tension element (12) is a thread or wire having a U-shaped profile with two U-legs, and extends through an eyelet or hook to or near the adjustable counter-guide.

7. Device according to claim 1, wherein the guiding part (5) is reinforced by at least one other parallel and/or at least section-wise curved brace (13) which extends in the use position on a side of the membrane (6) facing away from the heart wall (3) and which engage the membrane from behind or below and support the membrane.

8. Device according to claim 7, wherein the brace is arranged in a plane off-set relative to a surface of the membrane body (6).

9. Device according to one claim 1, further comprising a second guiding part (5) with at least one membrane body (6), the guiding parts being adapted to be located in the use position so that the heart wall (3) is arranged therebetween, and the two guiding parts (5) are connected to each other in the use position in a location adapted to be outside of the heart (4), such that the guiding parts and the membrane bodies (6) thereon are pressed together and therefore are adapted to be pressed from both sides onto the heart wall (3).

10. Device according to claim 9, wherein the two guiding parts (5) are connected in a tweezer-like manner and are arranged close to each other in the original position and can be spread apart for insertion in the use position against a restoring force.

11. Device according to claim 10, wherein the two guiding parts (5) are hinged outside of the region acted upon by the membrane bodies (6) in the use position at a region adapted to be located outside the heart (4), by a connecting device (16), and can be spread apart on a side of the connecting device (16) facing away from the membrane bodies (6) to bring the membrane bodies (6) closer to one another.

12. Device according to claim 11, wherein at least one spreading element (18) is located at regions of the guiding parts (5) that project past the connecting device (16) to spread open and fix a desired spread and provide an increased spreading force so that a pressure force acting on the membrane bodies (6) can be adjusted in a direction towards the connecting position (16).

13. Device according to claim 1, wherein the membrane body (6) is formed of an elastic material.

14. Device according to claim 7, wherein the at least one brace comprises two of the braces, with the braces being arranged on both sides of the guiding part (5),
